(19) United States
(12) Patent Application Publication
(10) Pub. No.: US 2025/0364099 A1
(43) Pub. Date: Nov. 27, 2025

(54) INFORMATION TRANSMISSION SYSTEM, INFORMATION TRANSMISSION METHOD, AND RECORDING MEDIUM

(71) Applicants: Sachika Tashiro, Kanagawa (JP); Youhei Niitsuma, Kanagawa (JP); Hirotaka Hayashi, Kanagawa (JP); Yoshihide Sugiyama, Kanagawa (JP); Hiroshi Fujie, Kanagawa (JP); Manabu Yamanaka, Kanagawa (JP); Takato Mochizuki, Kanagawa (JP); Yu Yamaya, Kanagawa (JP)

(72) Inventors: Sachika Tashiro, Kanagawa (JP); Youhei Niitsuma, Kanagawa (JP); Hirotaka Hayashi, Kanagawa (JP); Yoshihide Sugiyama, Kanagawa (JP); Hiroshi Fujie, Kanagawa (JP); Manabu Yamanaka, Kanagawa (JP); Takato Mochizuki, Kanagawa (JP); Yu Yamaya, Kanagawa (JP)

(73) Assignee: Ricoh Company, Ltd., Tokyo (JP)

(21) Appl. No.: 19/192,131

(22) Filed: Apr. 28, 2025

(30) Foreign Application Priority Data
May 22, 2024 (JP) 2024-083094

(51) Int. Cl. G16H 20/10

(52) U.S. Cl. CPC G16H 20/10

(57) ABSTRACT

An information transmission system includes a first information processing apparatus disposed in a first facility and a second information processing apparatus disposed in a second facility. The first information processing apparatus includes first circuitry. The first circuitry generates a code image in which a medication-related information group including medication-related information relating to a plurality of individuals taking medication is encoded, and forms the generated code image on a recording medium to be delivered from the first facility to the second facility. The second information processing apparatus includes second circuitry. The second circuitry acquires the medication-related information group from a captured image obtained by capturing an image of the code image on the recording medium delivered from the first facility to the second facility.

### CROSS-REFERENCE TO RELATED APPLICATIONS

This patent application is based on and claims priority pursuant to 35 U.S.C. § 119(a) to Japanese Patent Application No. 2024-083094, filed on May 22, 2024, in the Japan Patent Office, the entire disclosure of which is hereby incorporated by reference herein.

### BACKGROUND

### Technical Field

Embodiments of the present disclosure relate to an information transmission system, an information transmission method, and a recording medium.

### Related Art

A background information transmission system transmits medication-related information relating to an individual taking medication from a first information processing apparatus disposed in a first facility to a second information processing apparatus disposed in a second facility.

In a background information transmission system, for example, a mobile phone (first information processing apparatus) owned by a patient sends an electronic mail (e-mail) to a terminal (second information processing apparatus) in a dispensing pharmacy to transmit an item on a prescription (medication-related information) from the location where the patient is present to the dispensing pharmacy. In this system, the patient captures an image of a quick response (QR) Code™ on the prescription using an imaging device of the mobile phone and creates a message using the data in a predetermined prescription e-mail format and items on the prescription (such as the insurance number, name, and date of birth of the patient, and the name and quantity of the medicine) acquired from the captured image of the QR Code™ When the terminal in the dispensing pharmacy receives the e-mail sent from the mobile phone, a pharmacist in the dispensing pharmacy refers to the e-mail displayed on a display of the terminal and prepares the medicine before the patient arrives.

### SUMMARY

According to an embodiment of the present disclosure, an information transmission system includes a first information processing apparatus disposed in a first facility and a second information processing apparatus disposed in a second facility. The first information processing apparatus includes first circuitry. The first circuitry generates a code image in which a medication-related information group including medication-related information relating to a plurality of individuals taking medication is encoded, and forms the generated code image on a recording medium to be delivered from the first facility to the second facility. The second information processing apparatus includes second circuitry. The second circuitry acquires the medication-related information group from a captured image obtained by capturing an image of the code image on the recording medium delivered from the first facility to the second facility.

According to an embodiment of the present disclosure, an information transmission method is a method for transmitting medication-related information relating to a plurality of individuals taking medication from a first information processing apparatus disposed in a first facility to a second information processing apparatus disposed in a second facility and includes generating, by the first information processing apparatus, a code image in which a medication-related information group including the medication-related information relating to the plurality of individuals taking medication is encoded, forming, by the first information processing apparatus, the generated code image on a recording medium to be delivered from the first facility to the second facility, capturing, by the second information processing apparatus, an image of the code image on the recording medium delivered from the first facility to the second facility, and acquiring, by the second information processing apparatus, the medication-related information group from the captured image.

According to an embodiment of the present disclosure, a non-transitory recording medium stores a plurality of instructions which, when executed by one or more processors, causes the one or more processors to perform an information transmission method for transmitting medication-related information relating to a plurality of individuals taking medication from a first information processing apparatus disposed in a first facility to a second information processing apparatus disposed in a second facility. The information transmission method includes generating, by the first information processing apparatus, a code image in which a medication-related information group including the medication-related information relating to the plurality of individuals taking medication is encoded, forming, by the first information processing apparatus, the generated code image on a recording medium to be delivered from the first facility to the second facility, capturing, by the second information processing apparatus, an image of the code image on the recording medium delivered from the first facility to the second facility, and acquiring, by the second information processing apparatus, the medication-related information group from the captured image.

### BRIEF DESCRIPTION OF THE DRAWINGS

A more complete appreciation of embodiments of the present disclosure and many of the attendant advantages and features thereof can be readily obtained and understood from the following detailed description with reference to the accompanying drawings, wherein:

FIG. 1 is a diagram schematically illustrating a medication assistance system using an information transmission system;

FIG. 2A is a schematic front view of a medicine dispensing apparatus included in the medication assistance system;

FIG. 2B is a schematic side view of the medicine dispensing apparatus illustrated in FIG. 2A;

FIG. 3A is a plan view of a single unit-dose medicine pack;

FIG. 3B is a side view of the unit-dose medicine pack of FIG. 3A viewed in a direction indicated by an arrow A of FIG. 3A;

FIG. 3C is a side view of a medicine pack bundle;

FIG. 3D is a view of a medicine pack strip in an example form;

FIG. 4 is an external perspective view of a medicine dispensing tray used in the medication assistance system, illustrating an example configuration of the medicine dispensing tray;

FIG. 5 is a perspective view of an example compartment box held in the medicine dispensing tray:

FIG. 6A is a longitudinal cross-sectional view of an example cartridge used in the medicine dispensing apparatus;

FIG. 6B is a bottom view of the cartridge illustrated in FIG. 6A;

FIG. 7 is a block diagram illustrating a configuration of an information processing apparatus in a dispensing pharmacy for a process of creating and printing a code image in which a database file is encoded;

FIG. 8 is a view of an example prescription;

FIGS. 9A and 9B (FIG. 9) are diagrams illustrating example content of the database file;

FIG. 10A is a view of an example code image creation screen displayed on a display of the information processing apparatus;

FIG. 10B is a view of an example code image creation screen displayed on the display of the information processing apparatus;

FIG. 11 illustrates a view of an example screen displayed on the display of the information processing apparatus in the dispensing pharmacy that executes a predetermined application program and a view of an example screen displayed on a display of a personal computer in a nursing facility that executes the same predetermined application program;

FIG. 12 is a block diagram illustrating a configuration of the personal computer in the nursing facility for a process of acquiring a database file from a code image printed on a recording medium;

FIG. 13A is a view of an example database file extraction screen displayed on the display of the personal computer;

FIG. 13B is a view of an example database file extraction screen displayed on the display of the personal computer;

FIG. 14A is a flowchart illustrating a processing flow when the information processing apparatus in the dispensing pharmacy encrypts the database file;

FIG. 14B is a flowchart illustrating a processing flow when the personal computer in the nursing facility decrypts read data to restore the encrypted database file; and

FIG. 15 is a diagram illustrating an example case where an identifier (ID) of the nursing facility is used as an encryption key for encryption and decryption.

The accompanying drawings are intended to depict embodiments of the present disclosure and should not be interpreted to limit the scope thereof. The accompanying drawings are not to be considered as drawn to scale unless explicitly noted. Also, identical or similar reference numerals designate identical or similar components throughout the several views.

### DETAILED DESCRIPTION

In describing embodiments illustrated in the drawings, specific terminology is employed for the sake of clarity. However, the disclosure of this specification is not intended to be limited to the specific terminology so selected and it is to be understood that each specific element includes all technical equivalents that have a similar function, operate in a similar manner, and achieve a similar result.

Referring now to the drawings, embodiments of the present disclosure are described below. As used herein, the singular forms “a,” “an,” and “the” are intended to include the plural forms as well, unless the context clearly indicates otherwise.

Embodiments of the present disclosure are described below with reference to the drawings. An information transmission system according to an embodiment of the present disclosure is a system for transmitting a medication-related information group including medication-related information relating to a plurality of individuals taking medication from a first information processing apparatus disposed in a first facility to a second information processing apparatus disposed in a second facility. Specifically, an information processing system disposed in a dispensing pharmacy, which is an example of a first facility, transmits database data (a database file), which includes a medication-related information group, to a medicine dispensing system in a nursing facility, which is an example of a second facility. For example, the information transmission system according to the present embodiment is used for a medication assistance system that assists medication assistants such as nurses and caregivers in hospitals or nursing facilities in preparing and dispensing medication.

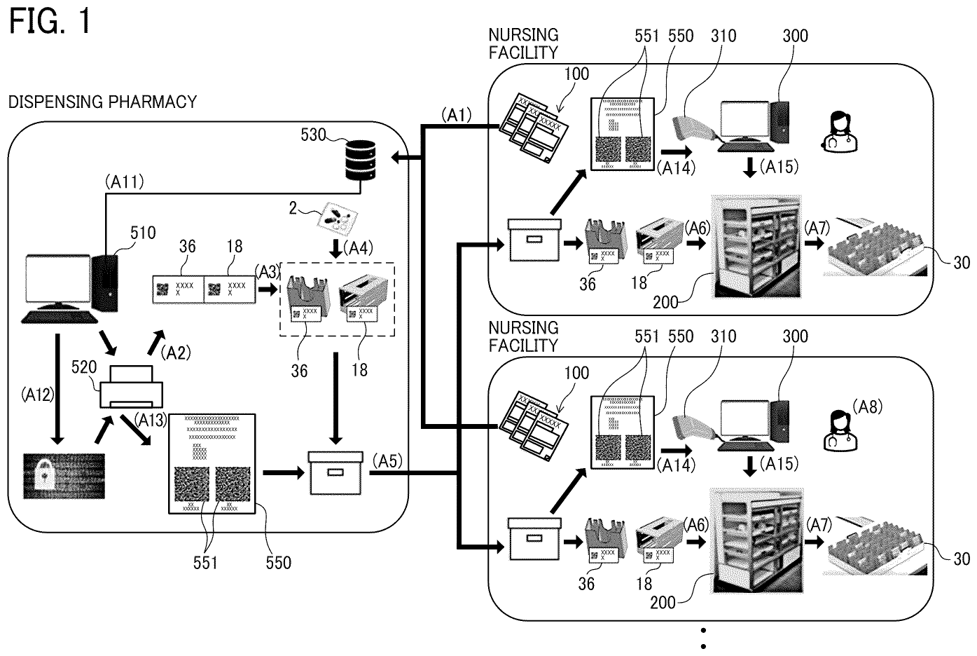

FIG. 1 is a diagram schematically illustrating a medication assistance system using the information transmission system according to the present embodiment. The medication assistance system according to the present embodiment includes, for example, an information processing system 500 and a medicine dispensing system. The information processing system 500 is disposed in a facility such as a dispensing pharmacy. In the present embodiment, as an example, the information processing system 500 is disposed in a dispensing pharmacy. The medicine dispensing system includes a medicine dispensing apparatus 200 and a personal computer 300, which are disposed in a facility such as a hospital or a nursing facility where individuals taking medication stay or reside. In the present embodiment, as an example, the medicine dispensing system is disposed in a nursing facility.

The information processing system 500 in the dispensing pharmacy functions as a first information processing apparatus and includes an information processing apparatus 510 and a printer 520, which are communicably connected to each other. The information processing apparatus 510 includes, for example, a personal computer. The printer 520 is an image forming device. The information processing system 500 disposed in the dispensing pharmacy includes a prescription computer 530. The information processing apparatus 510 is also communicably connected to the prescription computer 530.

The information processing apparatus 510 includes, for example, input devices such as a keyboard, a pointing device (e.g., a mouse), and a touch panel, a display device such as a display, an arithmetic device such as a central processing unit (CPU), storage devices such as a random-access memory (RAM), a read-only memory (ROM), and a solid-state drive (SSD), and a communication device for communicating with external devices (e.g., the printer 520 and devices that are communicably connected to the information processing apparatus 510 via communication lines). The information processing apparatus 510 functions as a label generator that generates label creation information from an acquired prescription information file including medication-related information. The label creation information is used to create labels 18 and 36, which are to be attached to medicines or medicine storages. The information processing apparatus 510 also functions as a code image generator that generates a code image from the acquired prescription information file including the medication-related information. The code image is an image in which a medication schedule file (database file) including a medication-related information group is encoded.

The printer 520 creates the labels 18 and 36 and prints a code image 551 on a recording medium 550 based on the label creation information and code image information generated by the information processing apparatus 510. The printer 520 may operate using any image formation method. In the printer 520, a label material on which no printing has not been made is set as a recording material. The printer 520 prints, on the label material, a print image specified by print data including the label creation information sent from the information processing apparatus 510. In the printer 520, the recording medium 550 on which the code image 551 including the encoded database file has not been printed is set. The printer 520 prints, on the recording medium 550, the code image 551 specified by the print data sent from the information processing apparatus 510.

Referring to FIG. 1, a hospital or a nursing facility provides a dispensing pharmacy with prescription information including medication-related information in A1. The medication-related information includes, for example, prescription information and information relating to an individual taking medication. The prescription information may be provided in paper or electronic format and includes, for example, the type of medicine and the timing of medication. The information relating to an individual taking medication includes, for example, the name of the nursing facility where the individual taking medication resides and the floor of the nursing facility at which the individual taking medication resides. In A2, a data file of the prescription information (prescription information file) is stored in the prescription computer 530, which is disposed in the dispensing pharmacy. The information processing apparatus 510, which is disposed in the dispensing pharmacy, extracts information from the stored prescription information file and prints the labels 18 and 36 using the printer 520.

Each of the labels 18 and 36 depicts a code image and a non-code image. The code image is an image in which at least part of the information extracted from the prescription information file (extracted information) is encoded. The non-code image includes a character string or a symbol (mark) indicating the content of at least part of the information extracted from the prescription information file (extracted information) to allow a human to visually recognize the content of the information on the non-code image. The code image may be a one-dimensional code image such as a barcode, a two-dimensional code image such as a quick-response (QR) Code™, or a three-dimensional code image. In the present embodiment, as an example, the code image is a two-dimensional code image. The labels 18 and 36 may depict both or one of the code image and the non-code image.

In the present embodiment, in A3, the labels 18 and 36 created by the information processing system 500 are attached to a cartridge 10 and a compartment box 34, respectively. The cartridge 10 is set in the medicine dispensing apparatus 200 in the nursing facility. The compartment box 34 is set in a medicine dispensing tray 30 in the nursing facility. Specifically, the labels 18 and 36 according to the present embodiment are attached to predetermined positions of the cartridge 10 and the compartment box 34, respectively. Alternatively, the labels 18 and 36 according to the present embodiment may be stored in label storage areas provided at the predetermined positions of the cartridge 10 and the compartment box 34, respectively, instead of being attached to the predetermined positions of the cartridge 10 and the compartment box 34, respectively.

The labels 18 and 36 may be applied to various objects and used in many ways other than being attached to the cartridge 10 of the medicine dispensing apparatus 200 or the compartment box 34 of the medicine dispensing tray 30.

In A4, in the dispensing pharmacy, a pharmacist stores medicine in the cartridge 10 to which the label 18 created by the information processing system 500 is attached. The medicine stored herein corresponds to the prescription information indicated by the label 18. In this example, a medicine pack bundle is stored in a stacked state in the cartridge 10. The medicine pack bundle includes a plurality of unit-dose medicine packs 2 joined together. Each unit-dose medicine pack 2 (hereinafter simply referred to as a “medicine pack”) includes one or more medicines packaged as a unit dose. In the present embodiment, the term “stored in a stacked state” means that the medicine packs 2 are stacked substantially horizontally or stacked flat.

In A5, the cartridge 10 storing the medicine (the medicine pack bundle including a plurality of medicine packs 2 joined together) is transported or delivered to the nursing facility together with the compartment box 34 to which the label 36 corresponding to the same prescription information is attached. In A6, the cartridge 10 and the compartment box 34 delivered to the nursing facility are set in the medicine dispensing apparatus 200, which is disposed in the nursing facility.

In A11, in the information processing system 500 in the dispensing pharmacy, the information processing apparatus 510 creates a database file including medication schedules from the prescription information file stored in the prescription computer 530, and stores the database file in a storage unit 512 of the information processing apparatus 510. This database file is used to control the medicine dispensing apparatus 200, which is disposed in the nursing facility.

In A13, the information processing apparatus 510, which is disposed in the dispensing pharmacy, creates the code image 551 in which the created database file is encoded, and then prints the code image 551 using the printer 520. In one example, the information processing apparatus 510 may encrypt the database file in A12 to more strongly prevent the leakage of personal information relating to a plurality of individuals taking medication, which is included in the database file. After that, in A13, the information processing apparatus 510 may create the code image 551 in which the encrypted database file is encoded, and then print the code image 551 using the printer 520.

The code image 551 in which the database file is encoded may be a one-dimensional code image such as a barcode, a two-dimensional code image such as a QR Code™, or a three-dimensional code image. In the present embodiment, as an example, the code image is a two-dimensional code image.

In the present embodiment, a recording medium on which the code image 551 including the encoded database file is printed may be, for example, a delivery item delivered from the dispensing pharmacy to the nursing facility or may be an item accompanying the delivery item. For example, when the code image 551 is printed on delivery items, the code image 551 may be printed on the cartridge 10 and the compartment box 34 to be delivered from the dispensing pharmacy to the nursing facility or on packaging materials for wrapping the cartridge 10 and the compartment box 34. For example, when the code image 551 is printed on an item accompanying the delivery items, the code image 551 may be printed on an accompanying document such as a statement of delivery of the cartridge 10 and the compartment box 34.

In this way, in A5, the recording medium 550 on which the code image 551 including the encoded database file has been printed is transported or delivered to the nursing facility together with the cartridge 10 and the compartment box 34. In another example, the recording medium 550 on which the code image 551 has been printed may be delivered to the nursing facility by mail separately from the delivery items such as the cartridge 10 and the compartment box 34.

The code image 551 printed on the recording medium 550 delivered to the nursing facility is read (captured) by a code scanner 310, which is an imaging device and connected to the personal computer 300 disposed in the nursing facility. In A14, the personal computer 300 reads and acquires the database file encoded in the code image 551 from the code image information (captured image) read by the code scanner 310. When the database file is encrypted, the personal computer 300 decrypts the database file to acquire the decrypted database file. In A15, the personal computer 300 sends the acquired database file to the medicine dispensing apparatus 200.

The medicine dispensing apparatus 200 automates, using the database file, a process of taking medicine out of each cartridge 10, which is provided for each individual taking medication, and storing the medicine in a corresponding one of the compartment boxes 34 for the individual taking medication, as described below. After the medicine dispensing apparatus 200 stores the medicine in each compartment box 34, each compartment box 34 is set in the medicine dispensing tray 30 in A7. In A8, a medication assistant carries the medicine dispensing tray 30 to the places where individuals taking medication reside and helps each individual to take the medicine stored in the corresponding one of the compartment boxes 34.

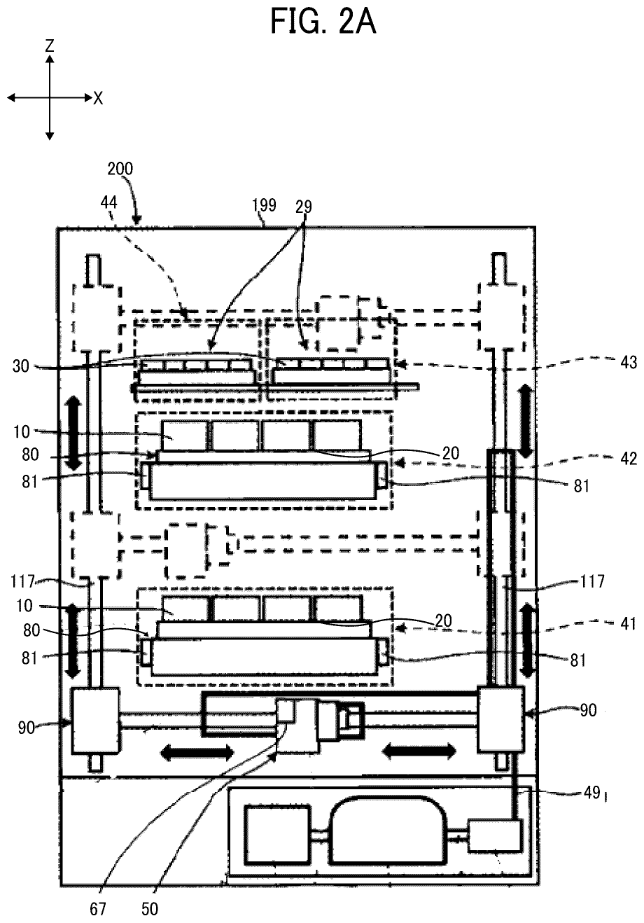

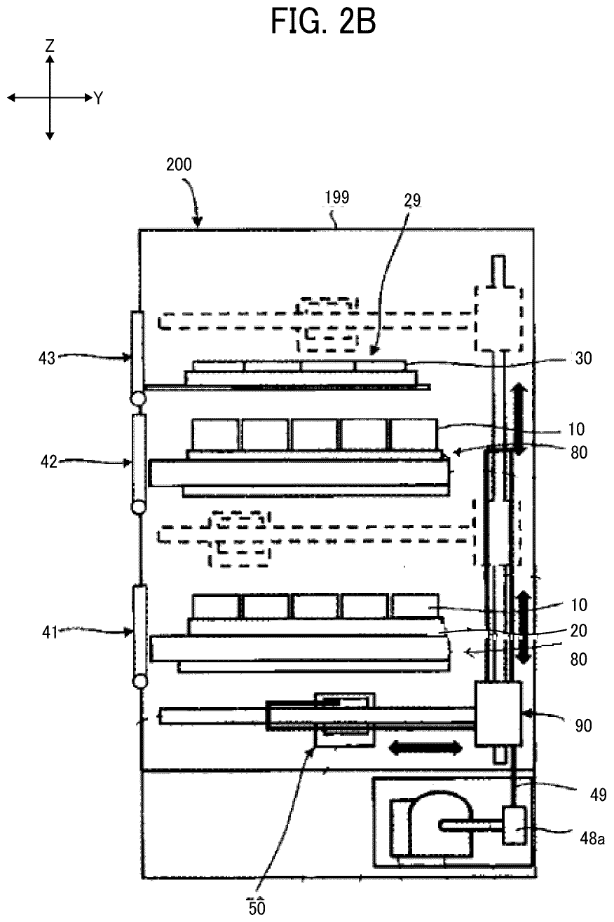

FIG. 2A is a schematic front view of the medicine dispensing apparatus 200, illustrating a configuration of the medicine dispensing apparatus 200. FIG. 2B is a schematic side view of the medicine dispensing apparatus 200, illustrating a side configuration of the medicine dispensing apparatus 200 illustrated in FIG. 2A. In FIGS. 2A and 2B, a left-right direction or a lateral direction (a width direction) of the medicine dispensing apparatus 200 is defined as an X direction, a front-back direction or a depth direction of the medicine dispensing apparatus 200 is defined as a Y direction, and an up-down direction or a longitudinal direction (a vertical direction) of the medicine dispensing apparatus 200 is defined as a Z direction.

As illustrated in FIGS. 2A and 2B, the medicine dispensing apparatus 200 includes cartridges 10, which are also referred to as first storages, cartridge trays 20, which are also referred to as second storages, drawers 80, medicine dispensing trays 30, which are medicine container holders, a carriage 50, which is also referred to as a taking-out device, a carrying device 90, and a first access gate 41 to a fourth access gate 44.

Some of the cartridges 10 are placed in one of the drawers 80 via a corresponding one of the cartridge trays 20 disposed in an uppermost portion of a body frame 199 of the medicine dispensing apparatus 200, while the other cartridges 10 are placed in another one of the drawers 80 via a corresponding one of the cartridge trays 20 disposed in a substantially central portion of the body frame 199 of the medicine dispensing apparatus 200. Each cartridge tray 20, which is integral with the corresponding drawer 80, positions and holds multiple cartridges 10 therein. Each cartridge tray 20 holds at least one cartridge 10. In the example illustrated in FIGS. 2A and 2B, cartridges 10 (4×5=20 cartridges 10) are placed and held in one drawer 80 (cartridge tray 20). For the sake of simplicity, the drawer 80 may be referred to as the cartridge tray 20. The cartridges 10 are detachably attached to the cartridge tray 20.

Each of the cartridges 10 is placed and stored in a corresponding one of the sections of the cartridge tray 20. The sections are each partitioned off by side walls and a bottom of the cartridge tray 20 wall so as to form a lattice structure. Each bottom wall of the cartridge tray 20 in which a corresponding cartridge 10 is stored has a rectangular through opening. The opening allows each medicine pack to be taken out from the bottom of the corresponding cartridge 10 using the elastic or freely-deformable nature of the medicine pack as described below.

The drawer 80 positions and holds the cartridge tray 20. The drawer 80 includes a pair of slide rails 81 attached to outer side walls of the drawer 80. The body frame 199 includes body rails such that the body rails are engaged with the slide rails 81 of the drawer 80. The engagement between the body rails and the slide rails 81 allows the drawer 80 in which the cartridge tray 20 is stored and held to be pulled out from the body frame 199.

Each medicine dispensing tray 30 is a medicine dispenser in which predetermined medicine packs carried by the carrying device 90 are arranged, and functions as a medicine container holder holding the compartment boxes 34, each of which serves as a medicine storage in which a medicine pack including one or more medicines is stored. As illustrated in FIG. 2A, the two medicine dispensing trays 30 are arranged above the cartridges 10 attached to the cartridge tray 20 in the uppermost drawer 80. The position at which each medicine dispensing tray 30 is disposed (location where medicine packs are supplied to be automatically dispensed to the medicine dispensing tray 30) is referred to as a medicine dispensing site 29.

The carriage 50 is a taking-out device or mechanism that takes out a predetermined medicine pack from the cartridge 10. The carrying device 90 functions as a carrying mechanism that carries the medicine pack taken out by the carriage 50 from the cartridge 10.

Each of the first access gate 41 and the second access gate 42 functions as a storage access gate, through which the cartridges 10 and the cartridge tray 20 are inserted into or removed from the body frame 199 via the drawer 80. When the cartridges 10 are inserted into and set in the body frame 199, the cartridges 10 are inserted from a corresponding one of the first access gate 41 and the second access gate 42. Each of the first access gate 41 and the second access gate 42 includes an openable door. When the cartridge 10 is inserted or removed, the door of the corresponding first access gate 41 or second access gate 42 is opened to allow the drawer 80 on which the cartridge tray 20 is set to be pulled out, so that the cartridge 10 or the cartridge tray 20 is insertable or removable.

The third access gate 43 and the fourth access gate 44 function as access gates for medicine dispensing, through which the two medicine dispensing trays 30 aligned on the uppermost stage of the medicine dispensing apparatus 200 are inserted into or taken out from the body frame 199. Each of the third access gate 43 and the fourth access gate 44 allows the corresponding medicine dispensing tray 30 to be taken out therefrom after the medicine packs are placed (hereinafter also referred to as “set” or “inserted”) in the medicine dispensing tray 30.

The two medicine dispensing trays 30 are disposed in the medicine dispensing apparatus 200. For example, one of the medicine dispensing trays 30 may be provided for one medication timing, such as morning, noon, evening, or before bedtime, and the other medicine dispensing tray 30 may be provided for another medication timing. The third access gate 43 is provided for one of the medicine dispensing trays 30, while the fourth access gate 44 is provided for the other medicine dispensing tray 30. This configuration allows one of the medicine dispensing trays 30 to be taken out while a medicine dispensing operation is carried out on the other medicine dispensing tray 30.

The medicine dispensing apparatus 200 has a temporal storage area 40 in its lower portion. When a medicine pack dispensed from the cartridge 10 is, for example, a wrong pack that is not supposed to be placed in the medicine dispensing tray 30, the wrong pack is temporarily stored in the temporal storage area 40.

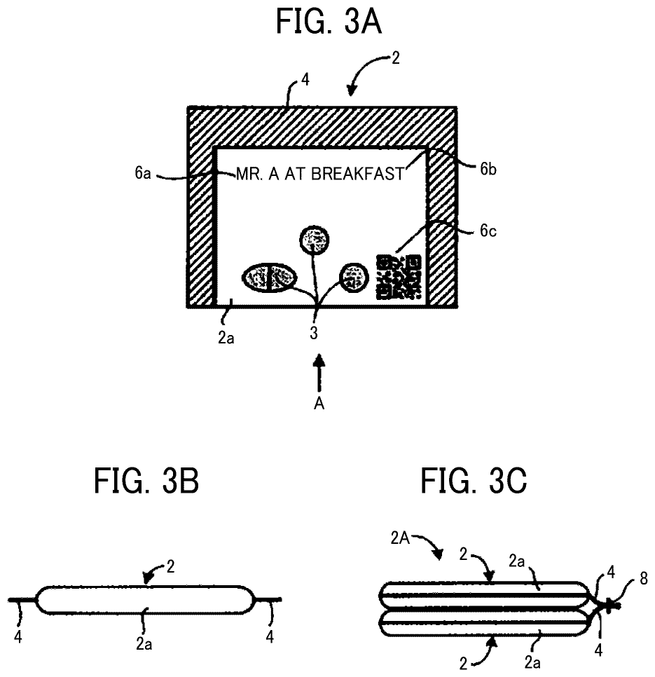

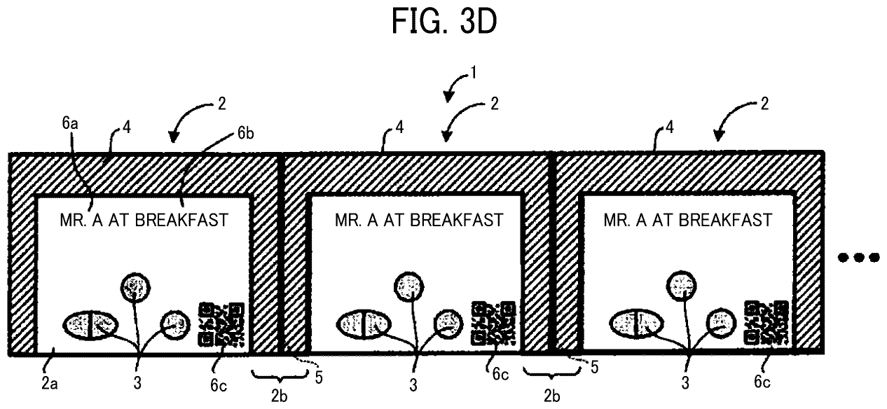

An overview of a unit-dose medicine pack (medicine pack) 2 is described with reference to FIGS. 3A to 3D. FIG. 3A is a schematic plan view of a single unit-dose medicine pack 2 in an example form. FIG. 3B is a side view of the unit-dose medicine pack 2 of FIG. 3A viewed in a direction indicated by an arrow A of FIG. 3A. FIG. 3C is a side view of a medicine pack bundle 2A in which a plurality of unit-dose medicine packs 2 is placed in the stacked state. FIG. 3D is a view of a medicine pack strip in an example form. For simplicity of illustration, medicines are omitted in FIGS. 3B and 3C.

One example form of a medicine pack is a single unit-dose medicine pack 2. Another example form of a medicine pack is a medicine pack bundle 2A in which a plurality of unit-dose medicine packs 2 (two unit-dose medicine packs 2 in FIG. 3C) is stacked in a stacking direction and joined together by, for example, stapling.

As illustrated in FIG. 3A, the single unit-dose medicine pack 2 includes, for example, a resin film, and medicines 3 such as capsules or tablets are packaged in the single unit-dose medicine pack 2. The unit-dose medicine pack 2 has a pouch portion 2a, which covers the medicines 3, and a pressure-bonded portion 4 where three sides of the unit-dose medicine pack 2 indicated by hatching in FIG. 3A are pressure-bonded or fused. The resin film is folded in half so that a space is created with the folded side serving as one side of the pouch portion 2a. In this space, the medicines 3 are inserted. The pressure-bonded portion 4 serves as a spill-prevention portion that prevents the medicines 3 from spilling out from the pouch portion 2a. In one example, each unit-dose medicine pack 2 includes one or more medicines 3 to be taken by an individual taking medication in a single dose.

Each unit-dose medicine pack 2 is formed (as a single pack) by a medicine packaging apparatus disposed in, for example, a pharmacy. A packaging sheet forming an exterior portion of a medicine pack 2 is a rolled long sheet. The packaging sheet is folded into two halves along a crease and rolled with the two halves overlapping with each other. The medicines 3 are inserted into a space between the two halves of the sheet. The three sides surrounding the medicines 3 other than the crease are sequentially sealed and divided by the pressure-bonded portion 4 to form unit-dose medicine packs 2 for multiple doses of medicines 3. In this way, a continuous sheet of unit-dose medicine packs 2 is formed for a predetermined number of doses of medicines 3. The continuous sheet of the unit-dose medicine packs 2 is referred to as a “medicine pack strip 1.” The medicine pack strip 1 illustrated in FIG. 3D includes a plurality of unit-dose medicine packs 2 that are coupled to each other as a strip. In the example illustrated in FIG. 3D, the medicine pack strip 1 includes unit-dose medicine packs 2 for three doses of medicines 3. The medicine pack strip 1 formed as described above is provided or sold at, for example, a pharmacy to a user. Examples of the user include an individual actually taking medicines packaged in a unit-dose medicine pack 2, a carer or assistant who helps or assists a user to take the medicines, and a staff member (including a pharmacist, a nurse, a caregiver, or a medication supporter) at various nursing facilities or medical facilities. For simplicity of illustration, the medicines 3 packaged in each medicine pack 2 have an identical form (e.g., capsules or tablets) in FIGS. 3A and 3D. In another example, the medicines 3 packaged in each medicine pack 2 may have different forms according to the usage and purpose of the medication for each user.

As illustrated in FIG. 3C, as an example, the medicine pack bundle 2A may be used. In one example, the medicine pack bundle 2A may be formed by joining a plurality of unit-dose medicine packs 2 (two unit-dose medicine packs 2 in FIG. 3C) together at the central portions of the three sides of the pressure-bonded portions 4 by stapling 8 (see FIG. 3C). In another example, the medicine pack bundle 2A may be formed by joining a plurality of unit-dose medicine packs 2 together by taping. In still another example, the medicine pack bundle 2A may be a bundle of Chinese medicine packs or press-through-package (PTP) sheets that are joined together.

The pouch portion 2a of the unit-dose medicine pack 2 has a surface to which first medication-related information is attached. The first medication-related information includes information relating to medication such as the name of an individual taking medication and the medication timing. More specifically, in the medicine pack 2 illustrated in FIG. 3A, the first medication-related information includes medication-related information 6a, medication-related information 6b, and medication-related information 6c. The medication-related information 6a includes a character string indicating the name of an individual taking the medicines 3 packaged in the medicine pack 2. The medication-related information 6b includes a character string indicating the medication timing at which the individual taking medication takes the medicines 3. The medication-related information 6c includes a two-dimensional code image such as a QR Code™ expressing the name of the individual taking medication and the medication timing. As an example, the two pieces of medication-related information 6a and 6b indicated by the character strings and the medication-related information 6c in the form of the two-dimensional code image are attached to the medicine pack 2 illustrated in FIG. 3A. However, the first medication-related information is not limited to the illustrated example. In one example, either the medication-related information 6a and the medication-related information 6b or the medication-related information 6c may be attached to the medicine pack 2. In another example, the medication-related information may be included in a barcode or a radio frequency identification (RFID) tag that stores tag information to be read through near-field communication. In still another example, a label including the medication-related information 6a, 6b, and 6c may be printed using the printer 520 and adhered to the surface of the pouch portion 2a of the unit-dose medicine pack 2.

The first medication-related information may further include the type of medicines (including the shapes of the medicines), the number of medicines packaged in the medicine pack 2, and information engraved in medicines themselves. The first medication-related information may include single information or a combination of a few pieces of information. For example, just to confirm who takes medication, it is sufficient that the first medication-related information includes single information such as the name of the individual taking medication. To prevent a missed dose, the first medication-related information may include a combination of a few pieces of information such as the name of the individual taking medication and the number of medicines or shapes of medicines packaged in the medicine pack 2 so that these pieces of information are confirmed at a time. Specifically, the first medication-related information includes at least one piece of information among the name of the individual taking medication, the medication timing at which the individual taking medication takes the medicines packaged in the medicine pack 2, and the types and the number of medicines packaged in the medicine pack 2.

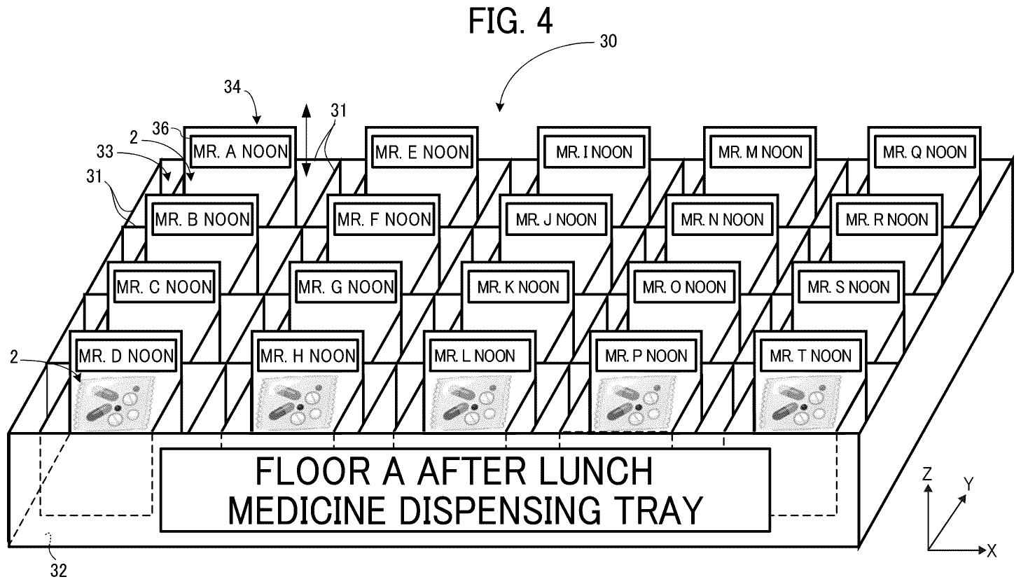

The medicine dispensing tray 30 is described below with reference to FIG. 4. FIG. 4 is an external perspective view of the medicine dispensing tray 30, illustrating an example configuration of the medicine dispensing tray 30. As illustrated in FIG. 4, the medicine dispensing tray 30 includes partition walls 31, which are partitioning members serving as a plurality of dividers for arranging predetermined medicine packs 2. Each set of four upright partition walls 31 partitions the medicine dispensing tray 30, thereby creating twenty sections 33. The twenty sections 33, which are formed in the medicine dispensing tray 30, may be represented as components of a matrix including five columns in an X direction (line-feeding direction) and four rows in a Y direction (character-feeding direction). With this configuration, the twenty sections 33 of the medicine dispensing tray 30 are specifically identified by the components and addresses of the matrix of five columns and four rows.

The medicine dispensing tray 30 further includes a bottom wall 32 on which the medicine packs 2 arranged in the sections 33 are placed. As described above, each medicine pack 2 is stored in a corresponding one of the sections 33 each partitioned off by a plurality of partition walls 31 and the shared bottom wall 32 of the medicine dispensing tray 30. With this configuration, each medicine pack 2 is individually manageable without being mixed with the other medicine packs 2 (for the other individuals taking medication).

In the medicine dispensing tray 30 illustrated in FIG. 4, the term “Floor A After lunch Medicine dispensing tray” is depicted on an external front wall surface of the medicine dispensing tray 30. This indicates, for example, that medicine packs 2 to be taken after lunch by a plurality of residents residing on the same floor A in a nursing facility are placed in the medicine dispensing tray 30.

The medicine dispensing tray 30 includes the compartment boxes 34, each of which is a medicine container detachably attached inside a corresponding one of the sections 33. The compartment boxes 34 store the medicines packs 2 in which medicines to be taken after lunch by twenty residents Mr. A to Ms. T residing in the nursing facility are packaged. Specifically, the medicine dispensing tray 30 stores each medicine pack 2 in a corresponding one of the compartment boxes 34 stored in the respective sections 33 each partitioned off by the plurality of partition walls 31.

In one example, the medicine packs 2 may be arranged in the medicine dispensing tray 30 without using the compartment boxes 34. In this case, each section 33 functions as a medicine container and each medicine pack 2 is directly stored in a corresponding one of the sections 33.

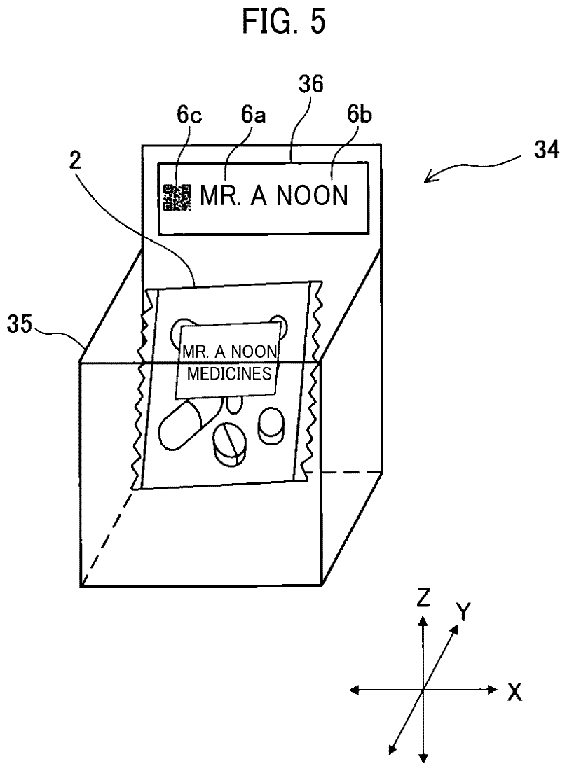

The compartment box 34 is described below with reference to FIG. 5. FIG. 5 is a perspective view of the compartment box 34 according to the present embodiment, which is held in the medicine dispensing tray 30. Each of the compartment boxes 34 is prepared for each resident (individual taking medication) and stores the unit-dose medicine pack 2 for the corresponding resident. For example, as illustrated in FIG. 5, the unit-dose medicine pack 2 to be taken by Mr. A after lunch is placed and stored in the corresponding compartment box 34 with character information “Mr. A, Noon” (first medication-related information) added.

The compartment box 34 has a box-shaped container structure including a material such as plastic (e.g., a plastic material) or paper. Specifically, as illustrated in FIG. 5, the compartment box 34 has a transparent structure 35 in the shape of a hollow cube or a hollow rectangular parallelepiped with an open top, and the internal space of the structure 35 functions as a medicine storage in which the medicine pack 2 is stored.

The structure 35 of the compartment box 34 has a rear wall higher than a front wall and side walls. The label 36 with the two pieces of character information 6a and 6b “Mr. A, Noon” and the two-dimensional code image 6c, which is a code image, is attached to an upper portion of the rear wall of the structure 35. The medicine dispensing apparatus 200 dispenses each unit-dose medicine pack 2 such that the medication information depicted on the label 36 such as the name of the individual taking medication and the medication timing matches the resident (individual taking medication) corresponding to the unit-dose medicine pack 2 to be stored in the compartment box 34 and the medication timing.

As a specific example, the label 36 on the compartment box 34 depicts the character information “Mr. A, Noon” and the two-dimensional code image 6c in which information associated (linked) with the information “Mr. A, Noon” is encoded. In this case, the information included (information encoded) in the two-dimensional code image 6c may be, for example, the actual character information depicted on the label 36, such as “Mr. A” and “Noon,” or identification information such as a unique identifier (ID) of the compartment box 34. When the information included in the two-dimensional code image 6c is identification information such as the unique ID, the medicine dispensing apparatus 200 refers to a database in which the identification information is associated with the character information such as “Mr. A” and “Noon” depicted on the label 36 so that the information included in the two-dimensional code image 6c is associated (linked) with the information on the label 36 on which the two-dimensional code image 6c is depicted.

The information included in the two-dimensional code image 6c on the label 36 attached to each compartment box 34 is read by, for example, a code reading device 67, which is included in the carriage 50. At this time, a coordinate of a X position and a coordinate of a Y position, which are the position information of the carriage 50, are associated (linked) with the information (the name of the individual taking medication and the medication timing) included in the two-dimensional code image 6c so that the medicine pack 2 taken out from the cartridge 10 is dispensed in an appropriate compartment box 34.

When each section 33 of the medicine dispensing tray 30 is allocated to each resident (individual taking medication), the label 36 including the two-dimensional code image 6c may be attached to a corresponding one of the sections 33 of the medicine dispensing tray 30, instead of each compartment box 34. Specifically, the plurality of sections 33 in the medicine dispensing tray 30 may be respectively allocated to a plurality of individuals taking medication at the same medication timing. When some individuals do not take medication at a specific medication timing, the medicine packs 2 for these individuals may be put aside without being placed at the specific medication timing.

In one example, the plurality of sections 33 of the medicine dispensing tray 30 may be allocated to different medication timings at which individuals take medication. For example, each section 33 may be allocated to each of the medication timings of each individual, such as morning, noon, evening, and before bedtime at which the individual takes medicines packaged in the medicine pack 2. In this example, the medicine dispensing tray 30 may be managed for each floor or room in which a plurality of individuals taking medication resides, and the medicine packs 2 for one day (or two or more days) may be dispensed in the medicine dispensing tray 30 in advance.

According to the above-described example, each section 33 is allocated to each medication timing such as morning, noon, evening, and before bedtime to prevent each individual taking medication from being medicated at a wrong timing. The configuration of the medicine dispensing tray 30 is not limited to the above-described configuration examples. The medicine dispensing tray 30 may have any of various other configurations according to a combination of individuals taking medication and medication timings.

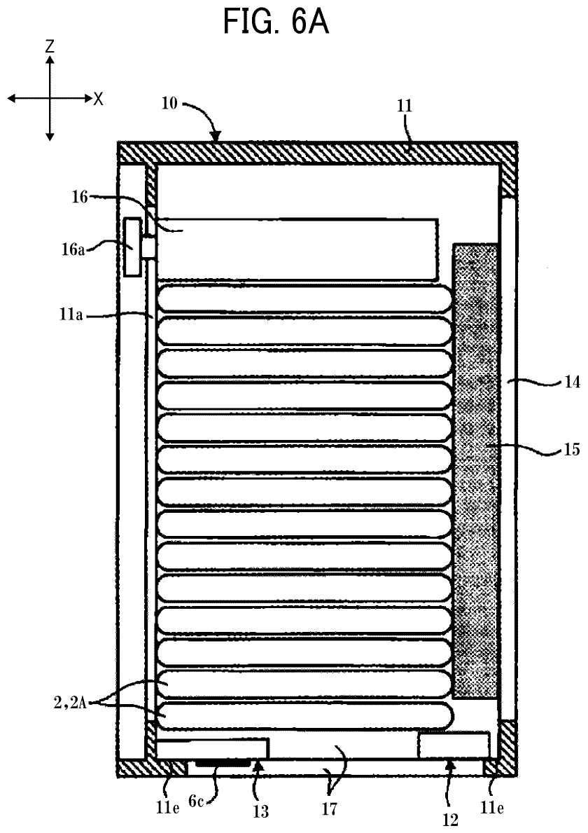

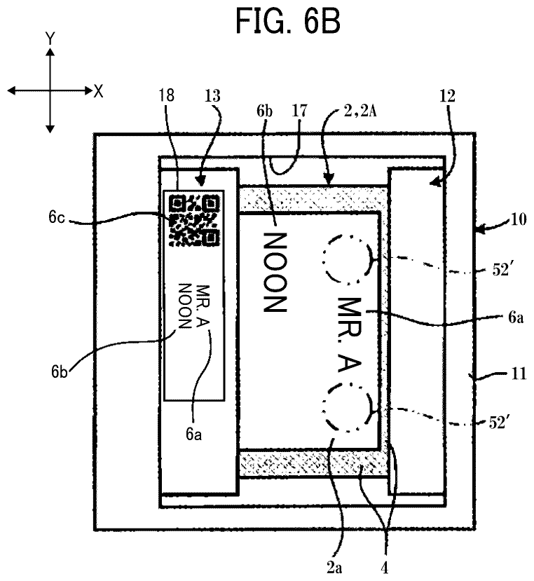

The cartridge 10 is described below with reference to FIGS. 6A and 6B. FIG. 6A is a longitudinal cross-sectional view of the cartridge 10. FIG. 6B is a bottom view of the cartridge 10 illustrated in FIG. 6A. For simplicity of illustration in the longitudinal cross-sectional view of FIG. 6A, the medicine packs 2 are schematically illustrated with the pressure-bonded portions 4 (see FIG. 3A) of the medicine packs 2 in the cartridge 10 omitted. Further, hatching of the cross-sections of supporters such as a right supporter 12 and a left supporter 13 is also omitted in FIG. 6A.

The cartridge 10 includes, for example, a case 11, a lid 14, a pack dispensing opening 17, a movable plate 16, a pack-posture retainer 15, the right supporter 12, and the left supporter 13. The right supporter 12 and the left supporter 13 serve as supporters.

The case 11 stores a plurality of medicine packs 2 or medicine pack bundles 2A. Hereinafter, the medicine packs 2 are taken as an example. The case 11 includes, for example, a resin and is integrally or separately mounted in the cartridge 10. The lid 14 allows the medicine packs 2 to be loaded into the case 11. The pack dispensing opening 17, which is formed at a lower portion or a bottom portion of the case 11, is an opening from which the medicine packs 2 inside the cartridge 10 are dispensed. The medicine packs 2 to be dispensed from the cartridge 10 by the carriage 50 pass through the pack dispensing opening 17.

The movable plate 16 prevents the medicine packs 2 from falling. After the first medicine pack 2 among the maximum number of medicine packs 2 storable in the case 11 is dispensed, the movable plate 16 moves the lowermost medicine pack 2 to the vicinity of the pack dispensing opening 17. The pack-posture retainer 15 retains the predetermined postures of the medicine packs 2. The right supporter 12 and the left supporter 13 support and retain the medicine packs 2 inside the case 11.

In the present embodiment, a dispensing mechanism from which the medicine packs 2 stored in the cartridge 10 are taken out by the carriage 50 is disposed at the lower portion or the bottom portion of the cartridge 10. Specifically, the dispensing mechanism includes the right supporter 12, the left supporter 13, and the pack dispensing opening 17. The right supporter 12 and the left supporter 13 serve as supporters or support members that support the medicine packs 2, which are to be dispensed from the cartridge 10, at multiple positions.

When the carriage 50 takes out a medicine pack 2 from the cartridge 10, the right supporter 12 and the left supporter 13 allow the medicine pack 2 to pass therebetween. When the carriage 50 does not take out any medicine pack 2 from the cartridge 10, the right supporter 12 and the left supporter 13 restrict the passage of the medicine packs 2 so that the medicine packs 2 remain stored and retained inside the case 11.

As described above, the right supporter 12 and the left supporter 13 are supporters that support and retain the medicine packs 2 inside the cartridge 10, and are disposed in a fixed state so as to allow the carriage 50 to stably take out each medicine pack 2 from the cartridge 10. The right supporter 12 and the left supporter 13 are fixed members that are fixed or attached to bottom-wall inner surfaces 11e at a right bottom wall end and a left bottom wall end of the pack dispensing opening 17, respectively.

The pack dispensing opening 17 allows suction pads 52 of the carriage 50 to pass therethrough to take out a medicine pack 2. The pack dispensing opening 17 also allows the suction pads 52 and the medicine pack 2 suctioned by the suction pads 52 to pass therethrough. Specifically, the pack dispensing opening 17, and the right bottom wall end and the left bottom wall end formed so as to surround the pack dispensing opening 17, function as a passage through which the suction pads 52 and the medicine pack 2 suctioned by the suction pads 52 pass.

In the cartridge 10 illustrated in FIG. 6B, positions 52′ (hereinafter also referred to as “suction pad positions”) at which the suction pads 52 suction a medicine pack 2 stored in the cartridge 10 are indicated with circular shapes with dashed-double-dotted lines in FIG. 6B. The right supporter 12 and the left supporter 13 support the medicine packs 2 inside the cartridge 10 such that the medicine packs 2 inside the cartridge 10 do not fall off from the pack dispensing opening 17. When the suction pads 52 suction the lowermost medicine pack 2 inside the cartridge 10 to take out the lowermost medicine pack 2, the two suction pads 52 suction the medicine pack 2 at two positions in the vicinity of opposite ends of the right supporter 12 in the Y direction. When the suction pads 52 suction the lowermost medicine pack 2 and takes out the medicine pack 2 from the cartridge 10, the two suction pads 52 pass through in the vicinity of the opposite ends of the right supporter 12 in the Y direction while suctioning and holding the medicine pack 2.

The pack-posture retainer 15 includes sponge rubber having appropriate elasticity. The movable plate 16 includes, for example, a resin or a metal. The pack-posture retainer 15 and the movable plate 16 retain appropriate postures of a plurality of medicine packs 2 stored in the case 11. As illustrated in FIG. 6A, the pack-posture retainer 15 and the movable plate 16 keep the postures of the plurality of medicine packs 2 orderly retained along the Z direction in a substantially horizontal state. In order to do so, the movable plate 16 moves downward in the Z direction inside the case 11 by its own weight, thereby assuredly moving at least one medicine pack 2 remaining in the case 11 toward the pack dispensing opening 17.

As illustrated in FIG. 6A, the case 11 has a long groove 11a on a side wall of the case 11. Specifically, the long groove 11a extends in the Z direction with a predetermined width in the X-direction. The movable plate 16 includes a shaft 16a with a flange at one end of the movable plate 16 in the X direction. The shaft 16a protrudes from the long groove 11a. The shaft 16a is guided along the long groove 11a in the Z direction so that the movable plate 16 retains the postures of the medicine packs 2 along the Z direction. As schematically illustrated in FIG. 6A, the medicine packs 2 inside the cartridge 10 are stacked on top of each other in layers in a substantially horizontally state and have inflating portions, omitted in FIG. 6A, on the left side of FIG. 6A in which medicines are packaged.

The medicine packs 2 are set in the case 11 such that the medicine packs 2 are sequentially stored in the upward direction from the pack dispensing opening 17 close to the right supporter 12 and the left supporter 13. The cartridge 10 may be replenished with the medicine packs 2 at, for example, a medical examination timing (e.g., once every two weeks) at which an individual (e.g., a resident) taking medication in a nursing facility is examined, or at a timing when the medicine packs 2 in the cartridge 10 run out. When one or more medicine packs 2 remain in the cartridge 10 at the time of the replenishment, additional medicine packs 2 are sequentially added from the back of the remaining medicine packs 2. In the present embodiment, the above-described setting or replenishment of the medicine parks 2 in the cartridge 10 is performed by, for example, a staff member of a nursing facility. In another embodiment, a storage may be implemented as the cartridge 10 in such a way that the setting or replenishment of the medicine parks 2 is performed automatically.

The lid 14 allows a staff member of a nursing facility, etc., to load the medicine packs 2 or unload the medicine packs 2 stored in the cartridge 10. As illustrated in FIG. 6A, the lid 14 has a length extending in the Z direction of the case 11 with a predetermined opening width.

As illustrated in FIG. 6B, the medicine packs 2 in the cartridge 10 are classified according to the timing at which the medicines are taken. In this example, one cartridge 10 stores the medicine packs 2 including medicines to be taken by Ms. A for 14 days at noon. When Ms. A takes the medicines in the morning, at noon, in the evening, and before bedtime, the total number of the cartridges 10 set for Ms. A is four. The classification is not limited to the example described above. For example, a single cartridge 10 may be set for each individual taking medication. In this case, the medicine packs 2 may be sequentially stored in the cartridge 10 in the upward direction from the pack dispensing opening 17 from which the medicine packs 2 are dispensed. Specifically, a medicine pack 2 including medicines to be taken in the morning of the first day is stored first, and then medicine packs 2 including medicines to be taken at noon, in the evening, and before bedtime of the first day are sequentially stored in this order on top of the medicine pack 2 for the morning of the first day. After that, medicine packs 2 including medicines to be taken in the morning, at noon, in the evening, and before bedtime of the second day are sequentially stored in this order on top of the medicine packs 2 for the first day.

In the present embodiment, as illustrated in FIG. 6B, the label 18 with the two-dimensional code image 6c depicted is attached to a bottom surface of the left supporter 13 in the vicinity of the pack dispensing opening 17. The code reading device 67, which is mounted on the carriage 50, reads the two-dimensional code image 6c on the label 18 to acquire medication-related information relating to medicine packs (such as medicine packs 2 or medicine pack bundles 2A) stored in the cartridge 10.

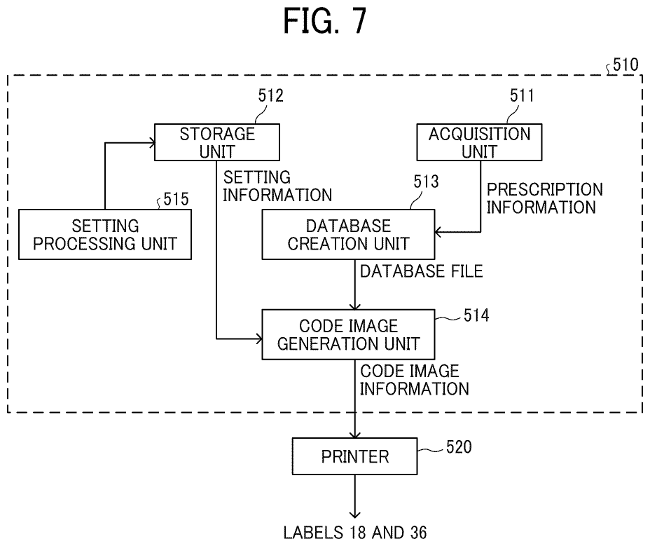

The information processing apparatus 510, which is included in the information processing system 500 and disposed in a dispensing pharmacy, is described below with reference to FIG. 7. FIG. 7 is a block diagram illustrating a configuration of the information processing apparatus 510 for a process of creating and printing the code image 551 in which a database file is encoded. The information processing apparatus 510 includes an acquisition unit 511, the storage unit 512, a database creation unit 513, a code image generation unit 514, and a setting processing unit 515.

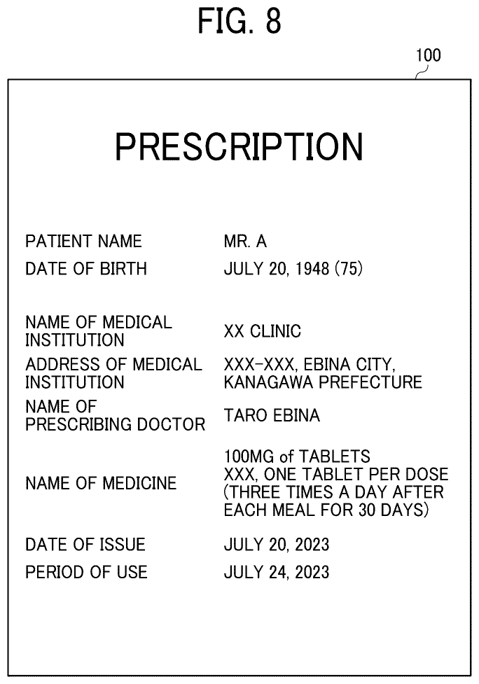

The acquisition unit 511 acquires medication-related information including prescription information. Specifically, an operator is provided with a prescription 100 for an individual taking medication from a nursing facility and inputs prescription information described in the prescription 100 using an input device of the information processing apparatus 510. One example of the prescription 100 is illustrated in FIG. 8. The acquisition unit 511 then acquires the prescription information input by the operator. The acquired prescription information is registered in a prescription information file stored in the prescription computer 530, which is disposed in the dispensing pharmacy. In the example illustrated in FIG. 8, the prescription 100 includes the name, the date of birth, and the age of the individual or patient taking medication, the name of the medical institution, the name of the doctor, the name of the medicine, the date of issue, and the period of use. The acquisition unit 511 may also read electronic data (prescription information file) including prescription information from a storage medium or receive the electronic data via a communication network to acquire the prescription information from the received prescription information file.

The acquisition unit 511 also acquires patient information as medication-related information. The patient information is information relating to the individual taking medication and owned by the dispensing pharmacy. The patient information includes, for example, the name of the nursing facility and the floor at which the individual taking medication resides. The acquired patient information is registered in the prescription information file stored in the prescription computer 530, together with the prescription information. Accordingly, the prescription information and the patient information, which are included in the medication-related information, are registered in association with each other in the prescription information file.

The storage unit 512 stores data used for a processing operation of the information processing apparatus 510. As described later, the storage unit 512 stores a setting file (setting data) for switching the operation of an application program (computer program) executed by a computer of the information processing apparatus 510.

The database creation unit 513 creates a database file for each nursing facility from the registered prescription information file. Each database file includes a medication schedule for each of a plurality of individuals taking medication who resides in a nursing facility. The database file created by the database creation unit 513 for each nursing facility is used to control the medicine dispensing apparatus 200, which is disposed in each nursing facility.

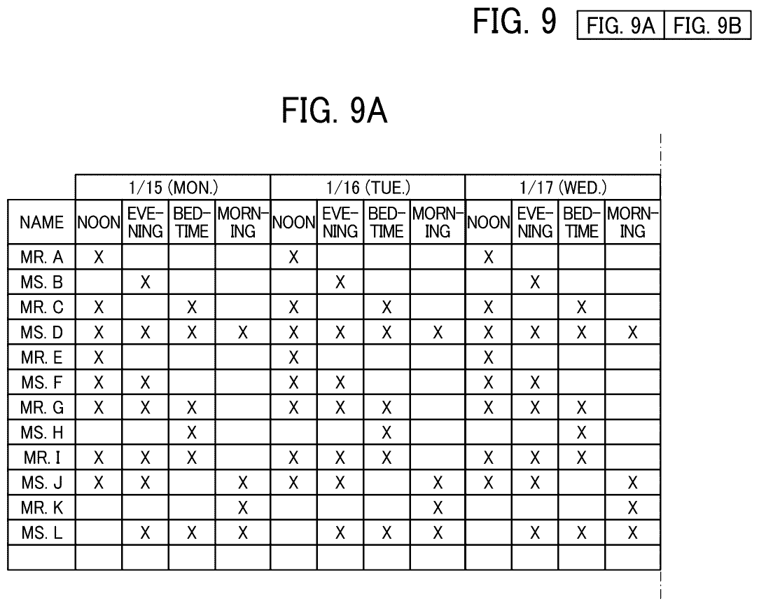

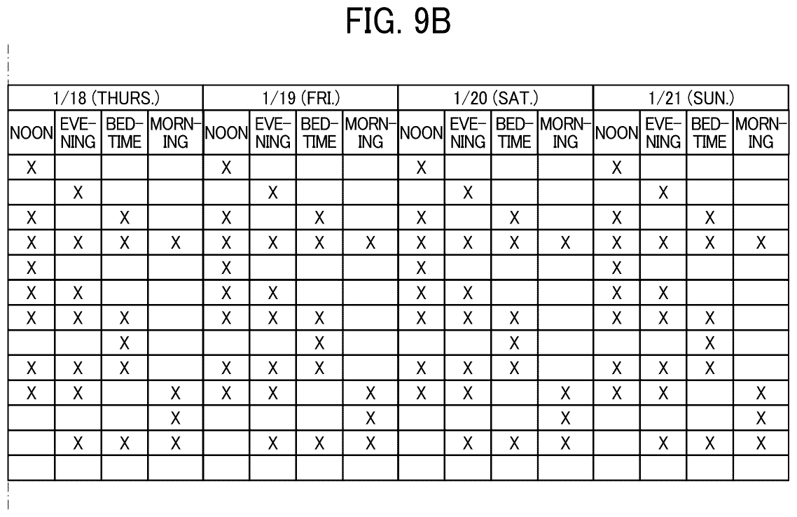

FIGS. 9A and 9B (hereinafter collectively referred to as FIG. 9) are diagrams illustrating example content of the database file. The database file illustrated in FIG. 9 is database data including medication schedules. Each medication schedule includes the medication dates and the medication timings (noon, evening, before bedtime, and morning in the example illustrated in FIG. 9) for each individual (e.g., a resident of the nursing facility) taking medication. The medication timings marked with crosses in the row of the medication schedule for each individual taking medication indicate the medication timings at which the individual taking medication takes medicines. The database file illustrated in FIG. 9 includes medication schedules for one week (from January 15 to January 21). In another example, the database file may include medication schedules for a period less than or more than one week. Further, as long as the database file includes medication schedules for two or more individuals taking medication, the database file may include medication schedules for any number of individuals taking medication.

The code image generation unit 514 creates the code image 551 in which the database file created by the database creation unit 513 is encoded. The information processing apparatus 510 sends the information included in the code image 551 generated by the code image generation unit 514 to the printer 520 as print data. The printer 520 prints the code image 551 specified by the print data on the recording medium 550.

In the present embodiment, there is a possibility that the amount of information included in a database file created by the database creation unit 513 exceeds the maximum amount of information that is encodable as a code image. In this case, the database file may be divided into two or more data to create two or more code images.

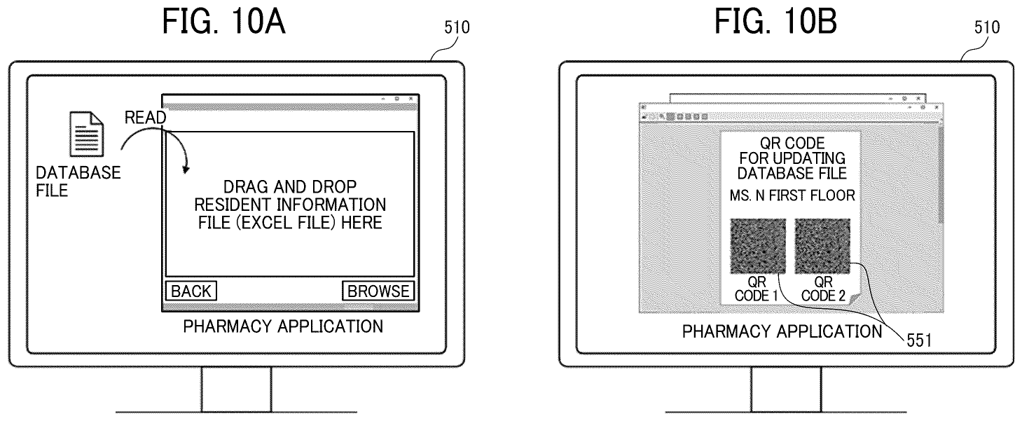

FIGS. 10A and 10B are views of an example code image creation screen displayed on a display of the information processing apparatus 510, which is disposed in a dispensing pharmacy. The computer of the information processing apparatus 510 executes a predetermined application program stored in the storage unit 512. At this time, the computer of the information processing apparatus 510 reads the setting file stored in the storage unit 512 to perform a processing operation for the dispensing pharmacy (a processing operation of the code image generation unit 514) according to the content of the setting file.

In the present embodiment, the predetermined application program stored in the storage unit 512 is a program package including a pharmacy application and a nursing-facility application. The pharmacy application includes a first program for causing the computer of the information processing apparatus 510 disposed in the dispensing pharmacy (first facility) to function as the code image generation unit 514. The nursing-facility application includes a second program for causing a computer of the personal computer 300 disposed in the nursing facility (second facility) to function as an information acquisition unit described below. The computer of the information processing apparatus 510 executes the pharmacy application included in the predetermined application program according to the content of the setting file stored in the storage unit 512.

When the computer of the information processing apparatus 510 executes the pharmacy application, an operation screen illustrated in FIG. 10A is displayed. An operator, such as a staff, of the dispensing pharmacy operates the input device of the information processing apparatus 510 on the operation screen to cause the pharmacy application to read a target database file. According to the content of the pharmacy application, the computer of the information processing apparatus 510 creates the code image 551 in which the read database file is encoded, and displays the processing result on the display of the information processing apparatus 510. An example of the processing result is depicted on the screen illustrated in FIG. 10B.

The setting processing unit 515 performs setting processing to add or change the setting file (setting data) stored in the storage unit 512 according to a predetermined setting command. For example, the setting file includes an item such as “For pharmacy.” When the item is “true” at the time of the execution of the predetermined application program, the pharmacy application is activated. When the item is “false,” the nursing-facility application is activated.

Using the setting file improves the convenience of the information transmission system according to the present embodiment since the pharmacy application (first program), which is used by the information processing apparatus 510 disposed in the dispensing pharmacy (an example of the first facility), and the nursing-facility application (second program), which is used by the personal computer 300 disposed in the nursing facility (an example of the second facility), are both included and distributed in a single program package.

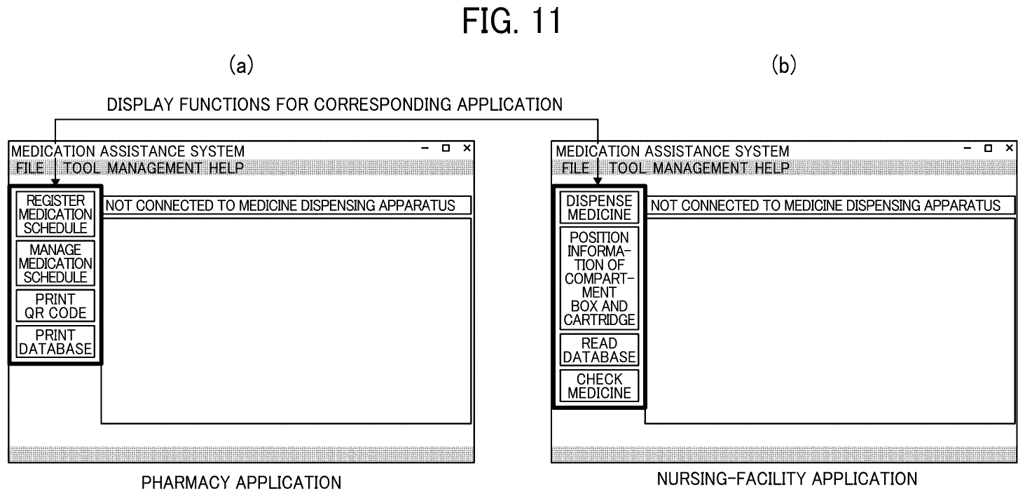

Furthermore, when the information transmission system according to the present embodiment is installed, the person in charge of the installation may set the content of the setting file by using the function of the setting processing unit 515. This eliminates the need for a user to switch between the pharmacy application and the nursing-facility application. With this setting, when the predetermined application program is executed on the information processing apparatus 510 in the dispensing pharmacy, the function menu of the pharmacy application is displayed as illustrated in part (a) of FIG. 11, but the function menu of the nursing-facility application is not displayed. Further, when the predetermined application program is executed on the personal computer 300 in the nursing facility, the function menu of the nursing-facility application is displayed as illustrated in part (b) of FIG. 11, but the function menu of the pharmacy application is not displayed.

With this configuration, while the application program is shared between the information processing apparatus 510 disposed in the dispensing pharmacy and the personal computer 300 disposed in the nursing facility, the information processing apparatus 510 and the personal computer 300 display the function menu dedicated to the dispensing pharmacy and the function menu dedicated to the nursing facility, respectively. This configuration, therefore, facilitates the operation of the operator.

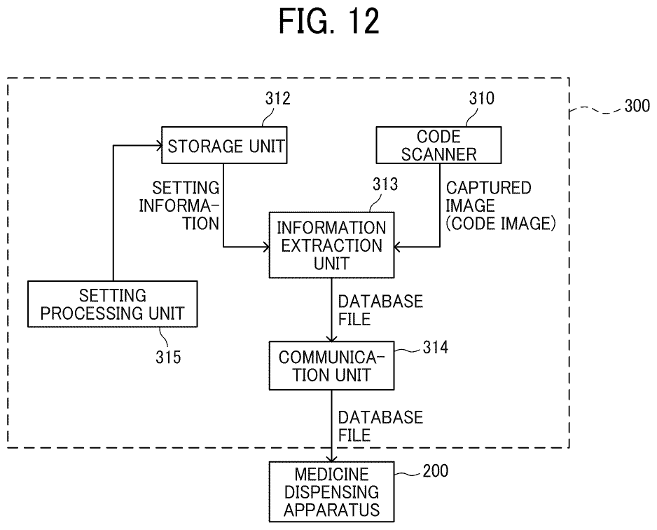

The personal computer 300, which is included in the medicine dispensing system in the nursing facility, is described with reference to FIG. 12. FIG. 12 is a block diagram illustrating a configuration of the personal computer 300 for a process of acquiring a database file from the code image 551 printed on the recording medium 550. The personal computer 300 includes the code scanner 310, which is an example of an imaging device, a storage unit 312, an information extraction unit 313, which is an example of an information acquisition unit, a communication unit 314, and a setting processing unit 315.

The code scanner 310 functions as an imaging device that captures an image of the code image 551 on the recording medium 550 delivered from the dispensing pharmacy to the nursing facility. The imaging device that captures an image of the code image 551 is not limited to the code scanner 310 and may be a camera or a device with a camera (such as a smartphone) that is connected to the personal computer 300 through wired or wireless communication.

The storage unit 312 stores data used for a processing operation of the personal computer 300. The storage unit 312 stores the setting file (setting data) for switching the operation of the application program (computer program) executed by the computer of the personal computer 300.

The information extraction unit 313 functions as the information acquisition unit that acquires the database file encoded in the code image 551 from the image of the code image 551 captured by the code scanner 310. The information extraction unit 313 may be provided as a function of the code scanner 310.

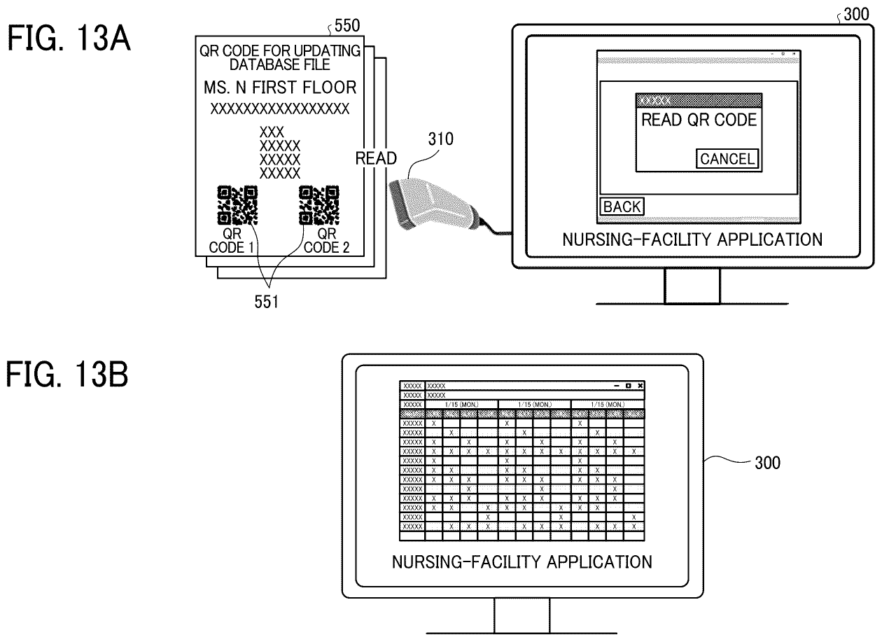

FIGS. 13A and 13B are diagrams illustrating an example of a database file extraction screen displayed on the display of the personal computer 300 in the nursing facility. The computer of the personal computer 300 executes the predetermined application program stored in the storage unit 312. At this time, the computer of the personal computer 300 reads the setting file stored in the storage unit 312 to perform a processing operation for the nursing facility (a processing operation of the information extraction unit 313) according to the content of the setting file.

In the present embodiment, the predetermined application program stored in the storage unit 312 is the program package including the pharmacy application and the nursing-facility application. The pharmacy application includes the first program for causing the computer of the information processing apparatus 510 disposed in the dispensing pharmacy (an example of the first facility) to function as the code image generation unit 514. The nursing-facility application includes the second program for causing the computer of the personal computer 300 disposed in the nursing facility (an example of the second facility) to function as the information acquisition unit. The computer of the personal computer 300 executes the nursing-facility application included in the predetermined application program according to the content of the setting file stored in the storage unit 312.

When the computer of the personal computer 300 executes the nursing-facility application, the operation screen illustrated in FIG. 13A is displayed. An operator, such as a staff, of the nursing facility views the operation screen and uses the code scanner 310 of the personal computer 300 to capture an image of the code image 551 on the recording medium 550 delivered from the dispensing pharmacy to the nursing facility. Accordingly, the nursing-facility application reads captured-image information obtained from the image of the code image 551 captured by the code scanner 310. According to the content of the nursing-facility application, the computer of the personal computer 300 extracts and acquires the database file encoded in the code image 551 from the read captured-image information. Accordingly, the content of the acquired database file is displayed on the display of the personal computer 300. An example of the content is displayed on the screen as illustrated in FIG. 13B.

The setting processing unit 315 performs setting processing to add or change the setting file (setting data) stored in the storage unit 312 according to a predetermined setting command. For example, the setting file includes an item such as “For pharmacy.” When the item is “true” at the time of the execution of the predetermined application program, the pharmacy application is activated. When the item is “false,” the nursing-facility application is activated.

The communication unit 314 sends the database file extracted (acquired) by the information extraction unit 313 to the medicine dispensing apparatus 200. The medicine dispensing apparatus 200 then dispenses medicines from each cartridge 10 prepared for each individual taking medication according to the medication schedules described in the received database file and performs a processing operation for storing the medicines in the respective compartment boxes 34 associated with the individuals taking medication.

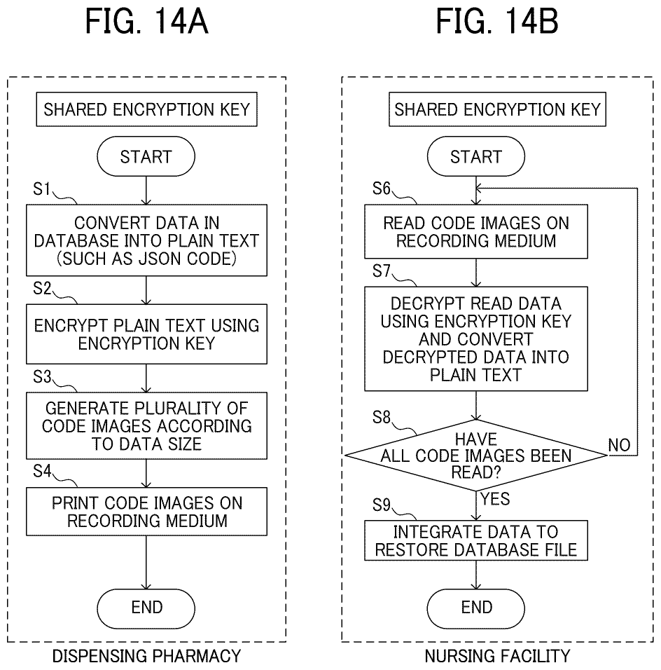

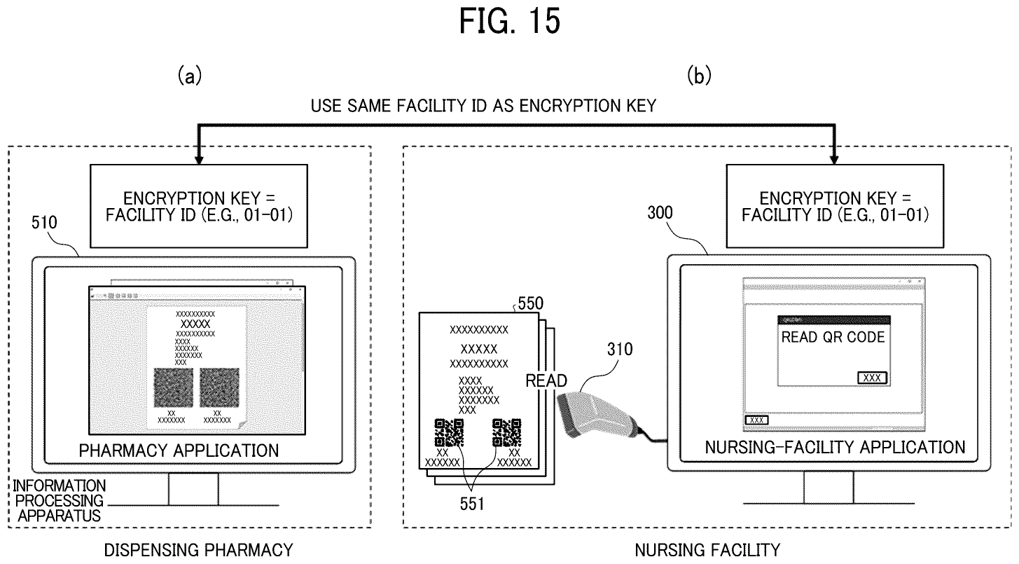

FIG. 14A is a flowchart illustrating a processing flow when the information processing apparatus 510 in the dispensing pharmacy encrypts the database file. FIG. 14B is a flowchart illustrating a processing flow when the personal computer 300 in the nursing facility decrypts (decodes) the encrypted database file to restore the database file. FIG. 15 is a diagram illustrating an example case where the ID of the nursing facility (second facility identification information) is used as an encryption key for encryption and decryption.

As a preliminary step, the ID (encryption key) of each nursing facility is registered in the pharmacy application that is executed on the information processing apparatus 510 in the dispensing pharmacy. In each nursing facility, the ID (encryption key) of the nursing facility is registered in the nursing-facility application that is executed on the personal computer 300.

The information processing apparatus 510, which is disposed in the dispensing pharmacy, performs processing in steps S1 to S4. In step S1, the information processing apparatus 510 in the dispensing pharmacy converts the data of a database file created by the database creation unit 513 into a plain text such as JavaScript object notation (JSON) code. After that, for example, an operator selects a nursing facility in which the database file is to be used by using the input device of the information processing apparatus 510. In response, in step S2, the information processing apparatus 510 encrypts the plain text using the ID of the selected nursing facility as an encryption key. In step S3, the information processing apparatus 510 encodes the encrypted data and generates one or more code images. The number of code images generated herein corresponds to the data size (the amount of information). In step S4, the information processing apparatus 510 prints the generated code images 551 on the recording medium 550 using the printer 520. The processing of the information processing apparatus 510 ends.

The personal computer 300, which is disposed in the nursing facility, performs processing in steps S6 to S9. The code scanner 310 of the personal computer 300 in the nursing facility reads (captures an image of) the code images 551 printed on the recording medium 550 delivered to the nursing facility. In step S6, the personal computer 300 reads the data encoded in the code images 551. In step S7, the personal computer 300 decrypts the read data using the ID of the nursing facility as the encryption key, and converts the data into a plain text. In step S8, the personal computer 300 determines whether or not all of the code images 551 have been read. When the personal computer 300 determines that all of the code images 551 have been read (Yes in step S8), the personal computer 300 integrates the data of the read code images that have been converted into a plain text, and then restores the database file in step S9. The personal computer 300 sends the database file acquired in this way to the medicine dispensing apparatus 200.

In this way, the data encoded in the code images 551 are encrypted using the ID of the nursing facility as the encryption key. This allows the personal computer 300 in the nursing facility associated with the encryption key to restore and acquire the database file from the code images 551. In other words, even when the recording medium 550 on which the code images 551 are printed is mistakenly delivered to a facility other than the target facility, ending up in the hands of a third party, the third party is unable to restore the database file. This configuration prevents the medication schedules (including the personal information of each individual taking medication) recorded in the database file from being leaked to the third party, thereby improving the security.

The embodiments described above are only examples and various aspects of the present disclosure attain respective effects as follows.

### Aspect 1

According to Aspect 1, an information transmission system is a system for transmitting medication-related information relating to a plurality of individuals taking medication from a first information processing apparatus (which includes, for example, the information processing apparatus 510) disposed in a first facility (e.g., a dispensing pharmacy) to a second information processing apparatus (which includes, for example, the personal computer 300) disposed in a second facility (e.g., a nursing facility) and includes the first information processing apparatus and the second information processing apparatus. The first information processing apparatus includes a code image generator (e.g., the code image generation unit 514) and an image forming device (e.g., the printer 520). The code image generator generates a code image (e.g., the code image 551) in which a medication-related information group (e.g., a database file) including the medication-related information relating to the plurality of individuals taking medication is encoded. The image forming device forms the code image generated by the code image generator on a recording medium (e.g., the recording medium 550) to be delivered from the first facility to the second facility. The second information processing apparatus includes an imaging device (e.g., the code scanner 310) and an information acquisition unit (e.g., the information extraction unit 313). The imaging device captures an image of the code image on the recording medium delivered from the first facility to the second facility. The information acquisition unit acquires the medication-related information group from the captured image obtained by the imaging device. In recent years, in some systems, a medication-related information group including medication-related information relating to a plurality of individuals taking medication is transmitted at a time from a first information processing apparatus disposed in a first facility to a second information processing apparatus disposed in a second facility. For example, medication-related information relating to a plurality of outpatients taking medication is transmitted from a hospital (an example of the first facility) from which the plurality of outpatients receives medical care to a dispensing pharmacy (an example of the second facility). In another example, medication-related information relating to a plurality of individuals taking medication is transmitted at a time from a dispensing pharmacy (an example of the first facility) to a facility (an example of the second facility) in which the plurality of individuals taking medication stays or resides. When sending medication-related information relating to a plurality of individuals taking medication, some systems send medication-related information relating to an individual taking medication one by one via electronic mail (e-mail), ending up in complicated procedures. Moreover, with these systems, just looking at the content of the e-mail allows anyone to see the medication-related information. Therefore, in order to prevent the leakage of personal information included in the medication-related information, an operation is conducted in such a complicated way that only limited personnel (person who has been authorized to deal with the medication-related information) is authorized to handle the e-mail. According to Aspect 1, the code image generator of the first information processing apparatus disposed in the first facility generates a code image in which a medication-related information group including medication-related information relating to a plurality of individuals taking medication is encoded, and the image forming device of the first information processing apparatus forms the generated code image on a recording medium. The recording medium on which the code image is formed is delivered from the first facility to the second facility. In the second information processing apparatus disposed in the second facility, the imaging device captures an image of the code image on the recording medium delivered from the first facility to the second facility, and the information acquisition unit acquires the medication-related information group from the captured image. According to Aspect 1, as described above, the code image is formed on the recording medium to be delivered from the first facility to the second facility and the medication-related information group is read from the code image. With this simplified method, the medication-related information group is transmitted from the first facility to the second facility. Moreover, even when someone looks at the code image on the recording medium, the person is unable to recognize the medication-related information encoded in the code image. Accordingly, even when a third party other than a person who is authorized to see the information handles the recording medium, personal information included in the medication-related information is not leaked to the third party. Therefore, the operation is conducted in such a simplified way that a third party is also allowed to handle the recording medium. In this way, according to Aspect 1, a medication-related information group including medication-related information relating to a plurality of individuals taking medication is transmitted with the simplified method while the leakage of the personal information is prevented.

### Aspect 2

According to Aspect 2, in the information transmission system of Aspect 1, the medication-related information group includes medication schedule information indicating a medication schedule for each of the plurality of individuals taking medication in at least one of the first facility and the second facility. According to Aspect 2, a medication schedule for each of a plurality of individuals taking medication is transmitted with the simplified method while the leakage of the personal information included in each medication schedule is prevented.

### Aspect 3

According to Aspect 3, in the information transmission system of Aspect 1 or 2, the recording medium includes a document (e.g., a statement of delivery) accompanying a delivery item delivered from the first facility to the second facility. Since the information transmission system eliminates the need to prepare a separate recording medium from the document accompanying the delivery item to be delivered from the first facility to the second facility, the operation is conducted with more simplified method.

### Aspect 4

According to Aspect 4, in the information transmission system of any of Aspects 1 to 3, the code image generator of the first information processing apparatus generates the code image in which the medication-related information group having been encrypted is encoded, and the information acquisition unit of the second information processing apparatus decrypts information read from the captured image obtained by the imaging device to acquire the medication-related information group. According to Aspect 4, even when the recording medium on which the code image is formed falls into the hands of a third party and the third party reads information from the code image, the third party is unable to acquire the medication-related information group encoded in the code image unless the third party successfully decrypts the medication-related information group. According to Aspect 4, therefore, the information transmission system achieves a higher level of security.

### Aspect 5

According to Aspect 5, in the information transmission system of Aspect 4, the code image generator generates the code image in which the medication-related information group having been encrypted using second facility identification information as an encryption key is encoded. The second facility identification information is identification information for identifying the second facility. In this way, according to Aspect 4, encryption and decryption are performed with the simplified method.

### Aspect 6

According to Aspect 6, in the information transmission system of any of Aspects 1 to 5, a computer included in the first information processing apparatus executes a first program to cause the code image generator of the first information processing apparatus to generate the code image. Further, a computer included in the second information processing apparatus executes a second program to cause the information acquisition unit of the second information processing apparatus to decrypt information read from the captured image to acquire the medication-related information group. The first program and the second program are included in the same program package. Since the same program package is provided and installed in the first information processing apparatus disposed in the first facility and the second information processing apparatus disposed in the second facility, the convenience of the information transmission system is improved.

### Aspect 7

According to Aspect 7, in the information transmission system of Aspect 6, the computer of the first information processing apparatus executes the first program included in the program package according to setting data of the program package to cause the computer of the first information processing apparatus to function as the code image generator. The computer of the second information processing apparatus executes the second program included in the program package according to setting data of the program package to cause the computer of the second information processing apparatus to function as the information acquisition unit. The first information processing apparatus and the second information processing apparatus include rewriting units (e.g., the setting processing units 515 and 315) to rewrite the respective setting data. The rewriting unit of the first information processing apparatus rewrites the setting data such that the first program dedicated to the first information processing apparatus is executed. Further, the rewriting unit of the second information processing apparatus rewrites the setting data such that the second program dedicated to the second information processing apparatus is executed. With this configuration, although the first information processing apparatus and the second information processing apparatus use the same program (program package), the first information processing apparatus and the second information processing apparatus execute their respective functions (a function of the first program and a function of the second program) without user operation.

### Aspect 8

According to Aspect 8, an information processing apparatus is used as the first information processing apparatus included in the information transmission system of any of Aspects 1 to 7. With the information processing apparatus according to Aspect 8, a medication-related information group including medication-related information relating to a plurality of individuals taking medication is transmitted with the simplified method while the leakage of the personal information is prevented.

### Aspect 9

According to Aspect 9, an information processing apparatus is used as the second information processing apparatus included in the information transmission system of any of Aspects 1 to 7. With the information processing apparatus according to Aspect 9, a medication-related information group including medication-related information relating to a plurality of individuals taking medication is transmitted with the simplified method while the leakage of the personal information is prevented.

### Aspect 10

According to Aspect 10, an information transmission method is a method for transmitting medication-related information relating to a plurality of individuals taking medication from a first information processing apparatus disposed in a first facility to a second information processing apparatus disposed in a second facility, and includes a code image generation process and an image formation process, which are performed by the first information processing apparatus, and an imaging process and an information acquisition process, which are performed by the second information processing apparatus. In the code image generation process, the first information processing apparatus generates a code image in which a medication-related information group including the medication-related information relating to the plurality of individuals taking medication is encoded. In the image formation process, the first information processing apparatus forms the code image generated in the code image generation process on a recording medium to be delivered from the first facility to the second facility. In the imaging process, the second information processing apparatus captures an image of the code image on the recording medium delivered from the first facility to the second facility. In the information acquisition process, the second information processing apparatus acquires the medication-related information group from the captured image obtained in the imaging process. With the information transmission method according to Aspect 10, a medication-related information group including medication-related information relating to a plurality of individuals taking medication is transmitted with the simplified method while the leakage of the personal information is prevented.

### Aspect 11

According to Aspect 11, a non-transitory recording medium stores a plurality of instructions which, when executed by a computer of a first information processing apparatus included in an information transmission system and disposed in a first facility, causes the computer to perform a code image generation process and an image formation process. The information transmission system is a system for transmitting medication-related information relating to a plurality of individuals taking medication from the first information processing apparatus to a second information processing apparatus disposed in a second facility. In the code image generation process, the computer of the first information processing apparatus generates a code image in which a medication-related information group including the medication-related information relating to the plurality of individuals taking medication is encoded. In the image formation process, the computer of the first information processing apparatus forms the code image generated in the code image generation process on a recording medium to be delivered from the first facility to the second facility. According to Aspect 11, a medication-related information group including medication-related information relating to a plurality of individuals taking medication is transmitted with the simplified method while the leakage of the personal information is prevented.

### Aspect 12

According to Aspect 12, a non-transitory recording medium stores a plurality of instructions which, when executed by a computer of a second information processing apparatus included in an information transmission system and disposed in a second facility, causes the computer to perform an information acquisition process. The information transmission system is a system for transmitting medication-related information relating to a plurality of individuals taking medication from a first information processing apparatus disposed in a first facility to the second information processing apparatus. In the information acquisition process, the computer of the second information processing apparatus acquires a medication-related information group from a captured image obtained by capturing, using an imaging device, an image of a code image formed on a recording medium delivered from the first facility to the second facility. The code image is an image in which the medication-related information group including the medication-related information relating to the plurality of individuals taking medication is encoded. According to Aspect 12, a medication-related information group including medication-related information relating to a plurality of individuals taking medication is transmitted with the simplified method while the leakage of the personal information is prevented.

The above-described embodiments are illustrative and do not limit the present invention. Thus, numerous additional modifications and variations are possible in light of the above teachings. For example, elements and/or features of different illustrative embodiments may be combined with each other and/or substituted for each other within the scope of the present invention. Any one of the above-described operations may be performed in various other ways, for example, in an order different from the one described above.

The functionality of the elements disclosed herein may be implemented using circuitry or processing circuitry which includes general purpose processors, special purpose processors, integrated circuits, application-specific integrated circuits (ASICs), field-programmable gate arrays (FPGAs), and/or combinations thereof which are configured or programmed, using one or more programs stored in one or more memories, to perform the disclosed functionality. Processors are considered processing circuitry or circuitry as they include transistors and other circuitry therein. In the disclosure, the circuitry, units, or means are hardware that carry out or are programmed to perform the recited functionality. The hardware may be any hardware disclosed herein which is programmed or configured to carry out the recited functionality.

There is a memory that stores a computer program which includes computer instructions. These computer instructions provide the logic and routines that enable the hardware (e.g., processing circuitry or circuitry) to perform the method disclosed herein. This computer program can be implemented in known formats as a computer-readable storage medium, a computer program product, a memory device, a record medium such as a CD-ROM or DVD, and/or the memory of an FPGA or ASIC.

The recording medium is one or more media readable by a computer, which stores one or more programs executable by the computer. The computer executing the program to perform certain processing and the computer having the recording medium may be different computers.

The recording medium includes, for example, electronic recording medium, a magnetic recording medium, an optical recording medium, an electromagnetic recording medium, and a semiconductor recording medium, or any suitable combination of the foregoing. More specific examples of the recording medium include a read-only memory (ROM), a random access memory (RAM), an erasable programmable read-only memory (EPROM), a static random access memory (SRAM), a non-volatile RAM (NVRAM), a dynamic RAM (DRAM), a hard disk, a flash memory, a compact disc ROM (CD-ROM), a digital versatile disk ROM (DVD-ROM), a memory stick, and a floppy disk.

The computer may read one or more programs from the recording medium. The reading the programs is not limited to reading the programs from the recording medium that is provided in the computer or the recording medium removable from the computer via an interface, but may include downloading the programs from another computer onto the computer. For example, the programs may be stored in a recording medium of the other computer operating as a server. The other computer may read the programs from the recording medium, and transmit the read programs to the computer executing such program via a network such as the Internet or a local area network. The other computer receives the programs via the network, and stores the programs in the recording medium of the computer. In this way, the computer downloads the programs from the recording medium of the other computer via the network, and installs the downloaded program in the recording medium of the computer.

The recording medium includes one or more recording media provided in the computer executing the programs, one or more removable recording media communicable with a computer via an interface, and one or more recording media provided at one or more other computers that provide the computer executing the programs with the programs. The programs include a set of computer instructions, which may execute entirely on one computer, or partly on one computer and partly on another computer. The programs may be recorded on a recording medium that is readable by the computer so as to configure the computer to execute each function of the above-described embodiments. The computer reads the programs stored in the recording medium to perform each processing of the above-described embodiments.

1. An information transmission system comprising:
a first information processing apparatus disposed in a first facility; and
a second information processing apparatus disposed in a second facility,
the first information processing apparatus including first circuitry configured to:
generate a code image in which a medication-related information group including medication-related information relating to a plurality of individuals taking medication is encoded; and
form the generated code image on a recording medium to be delivered from the first facility to the second facility,
the second information processing apparatus including second circuitry configured to acquire the medication-related information group from a captured image obtained by capturing an image of the code image on the recording medium delivered from the first facility to the second facility.

2. The information transmission system according to claim 1, wherein the medication-related information group includes medication schedule information indicating a medication schedule for each of the plurality of individuals taking medication in at least one of the first facility or the second facility.

3. The information transmission system according to claim 1, wherein the recording medium includes a document accompanying a delivery item delivered from the first facility to the second facility.

4. The information transmission system according to claim 1,
wherein the first circuitry of the first information processing apparatus is configured to generate the code image in which the medication-related information group having been encrypted is encoded, and
the second circuitry of the second information processing apparatus is configured to decrypt information read from the captured image to acquire the medication-related information group.

5. The information transmission system according to claim 4, wherein the first circuitry is configured to generate the code image in which the medication-related information group having been encrypted using second facility identification information as an encryption key is encoded, the second facility identification information being identification information for identifying the second facility.

6. The information transmission system according to claim 1,
wherein the first circuitry of the first information processing apparatus is configured to execute a first program to generate the code image,
the second circuitry of the second information processing apparatus is configured to execute a second program to decrypt information read from the captured image to acquire the medication-related information group, and
the first program and the second program are included in a same program package.

7. The information transmission system according to claim 6,
wherein the program package includes setting data,
the first circuitry of the first information processing apparatus is configured to execute the first program included in the program package according to the setting data of the program package,
the second circuitry of the second information processing apparatus is configured to execute the second program included in the program package according to the setting data of the program package, and
the first circuitry and the second circuitry are configured to rewrite the respective setting data.

8. An information transmission method for transmitting medication-related information relating to a plurality of individuals taking medication from a first information processing apparatus disposed in a first facility to a second information processing apparatus disposed in a second facility, the information transmission method comprising:
generating, by the first information processing apparatus, a code image in which a medication-related information group including the medication-related information relating to the plurality of individuals taking medication is encoded;
forming, by the first information processing apparatus, the generated code image on a recording medium to be delivered from the first facility to the second facility;
capturing, by the second information processing apparatus, an image of the code image on the recording medium delivered from the first facility to the second facility; and
acquiring, by the second information processing apparatus, the medication-related information group from the captured image.

9. A non-transitory recording medium storing a plurality of instructions which, when executed by one or more processors, causes the one or more processors to perform an information transmission method for transmitting medication-related information relating to a plurality of individuals taking medication from a first information processing apparatus disposed in a first facility to a second information processing apparatus disposed in a second facility, the information transmission method comprising:
generating, by the first information processing apparatus, a code image in which a medication-related information group including the medication-related information relating to the plurality of individuals taking medication is encoded;
forming, by the first information processing apparatus, the generated code image on a recording medium to be delivered from the first facility to the second facility;
capturing, by the second information processing apparatus, an image of the code image on the recording medium delivered from the first facility to the second facility; and
acquiring, by the second information processing apparatus, the medication-related information group from the captured image.
